(19) United States
(12) Patent Application Publication
(10) Pub. No.: US 2005/0033088 A1
(43) Pub. Date: Feb. 10, 2005

(54) Process for the preparation of phenethylamine derivative, an intermediate of Venlafaxine hydrochloride

(75) Inventors: Buchi Reddy Reguri, Hyderabad (IN); Rajasekhar Kadaboina, Hyderabad (IN); Srinivas Reddy Gade, Hyderabad (IN); Babu Ireni, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/862,890

(22) Filed: Jun. 7, 2004

(30) Foreign Application Priority Data
Jun. 6, 2003 (IN) 460/MAS/2003

(51) Int. Cl. C07C 231/02

(52) U.S. Cl. 564/141

(57) ABSTRACT

The present invention relates to an improved process for the preparation of phenethylamine derivatives or salts by hydrogenation of phenylacetonitriles in the presence of heterogeneous palladium on carbon catalyst.

The present invention relates to an improved process for the preparation of phenethylamine derivatives or salts by hydrogenation of phenylacetonitriles in the presence of heterogeneous palladium on carbon catalyst.

Particularly the present invention relates to an improved process for the preparation of phenethylamine derivative of formula (X) or salts thereof 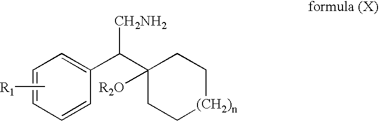
wherein,
R1 is H, OH, or unsubstantiated or substituted alkyl or alkoxy;
R2 is hydrogen or a substituent, which can be converted to hydrogen, and n is 0, 1 or 2.
   Or,

The compound of formula (X) where R₁ is an alkyl of straight chain or branched alkyl substituent, preferably C₁-C₄ alkyl, such as methyl, ethyl n-propyl, isopropyl, n-butyl, sec-butyl and tert-butyl.

Or,

The compound of formula (X) where R₁ is hydroxy, alkoxy of straight chain or branched alkoxy, preferably C₁-C₄ alkoxy, such as methoxy, ethoxy, n-propyl, isopropoxy, n-butoxy, sec-butoxy and ter-butoxy preferably methoxy.

It is preferred that R1 is boundled to the benzene ring at para-position.

R₂ is hydrogen, formyl or C₂-C₆ alkonyl, preferably hydrogen.

And n=0,1,2,3 preferably 1.

According to a preferred embodiment of the present invention the process for the preparation of acetate salt of the compound of formula (X) where,
R1 is methoxy, R2 is hydrogen and n is 1, an intermediate of Venalafaxine hydrochloride which is herein after referred as formula (1).

### BACKGROUND OF INVENTION

The compounds of formula (1) are known for being particularly useful as intermediates for preparing pharmaceutical active substances, which are central nervous system antidepressants, an important such substance is Venlafaxine. The preparation of this compound is described in U.S. Pat. No. 4,535,186.

In U.S. Pat. No. 6,350,912 the process for the preparation of Venlafaxine has been disclosed as one pot process. In the said patent compound of formula (1) has been disclosed by the reduction of corresponding cyano compound in the presence of Raney nickel followed by conversion to Venlafaxine without isolation of compound of formula (1).

In WO 0250017 the process for the preparation of the compound of formula (1) has been disclosed by the reduction of corresponding cyano compound in the presence of nickel or cobalt catalyst.

According to U.S. Pat. No. 4,535,186 Example 2, the commpound of formula (1) are prepared by hydrogenation in the presence of a rhodium catalyst. The use of this catalysts under high pressure poses severe drawbacks because of its hazardous nature. And also the use of this catalyst is inconvenient in the large-scale preparation of compound of formula (1).

Hence it is necessary to provide a simple and convenient process for the Synthesis of compound of formula (1).

It is the object of the present invention to provide a process for the preparation of compound of formula (1) with good purity, which meets economical demands.

It is the object of the present invention to provide a process for the preparation of compound of formula (1) with good purity, which meets economical demands.

The main object of the present invention is to provide a simple and convenient process for the synthesis of compound of formula (1) by using simple catalyst.

### SUMMARY OF THE INVENTION

Accordingly, the present invention provides an improved process for the preparation of 1-[2-amino-1-(4-methoxyphenyl)ethyl]cyclohexanol acetate (an intermediate of Venlafaxine hydrochloride) of formula (1), which comprises, by reduction of corresponding compound of formula (2) i.e 1-[cyano-(4-methoxyphenyl)methyl]cyclohexanol using palladium on carbon in the presence of organic acid. The compound of formula (1) further converted to Venlafaxine hydrochloride in high yields. Venlafaxine hydrochloride can be depicted by the formula (3).

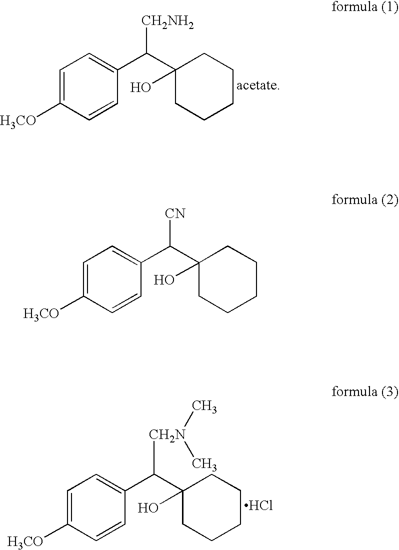

### DETAILED DESCRIPTION OF THE INVENTION

The present invention is directed to an improved process for synthesizing compound of formula (1) by reduction of compound of formula (2). The compound of formula (1) further converted to Venlafaxine hydrochloride of formula (3)

The synthetic pathway can be depicted in the following Scheme.

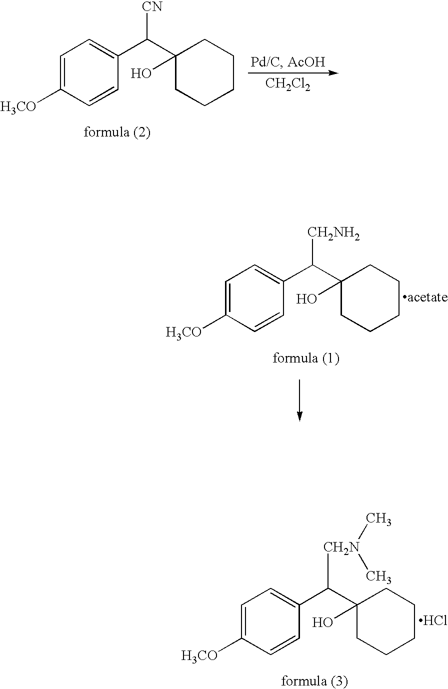

Accordingly an improved process for the preparation of compound of formula (1), which comprises:
a. reduction of 1-[cyano(4-methoxyphenyl)methyl)cyclohexanol of formula (2) with palladium on charcoal in an organic acid selected from formic acid, acetic acid or propionic acid, preferably acetic acid in an autoclave at a pressure of 5-25 kg/cm2 preferably 10-15 kg/cm2 at a temperature in the range of 30-75° C., preferably at 50-55° C. till the hydrogenation substantially complete;
b. filteration of the palladium catalyst from the reaction mass of step (a);
c. evaporating the filtrate of step (b) under reduced pressure;
d. suspending the solid type residue of step (c) in water and the resulted solution was basified by addition of aqueous ammonia;
e. extraction of the solution of step (d) with halogenated hydrocarbon solvents such as methylene chloride or chloroform preferably methylene chloride;
f. washing the organic solution of step (e) with water;
g. evaporation of the organic solvent from step (f) under reduced pressure to get residue;
h. dissolution of the residue from step (g) in C1-C4 alcohols preferably isopropanol;
i. cooling the mass of step (h) at 0-35° C. preferably at 0-5° C.;
j. filtration of separated solid from step (i) to get the desired compound of formula (1);
k. taken the low melting solid obtained from step (j) into ethyl acetate and added acetic acid at 0° C.;
l. filtered the separated solid from step (k), and dried at 45-60° C. preferably 55° C.

The obtained compound of formula (1) further converted to Venlafaxine hydrochloride of formula (3) without isolation of its freebase and without using any chromatographic techniques in high yields.

The improved process of the present invention for preparation of compound of formula (1) and further conversion to Venlafaxine Hydrochloride is hence directed to a more industrially suited, free from fire hazardous catalytic transformation, commercially viable and cost effective process having the following advantages over prior art references.
The present invention provides an alternative and simple two-stage method for preparation of Venlafaxine hydrochloride of formula (3). The first stage involves usage of commercially available and relatively less expensive palladium on carbon for reduction of compound of formula (2). The reduction is exemplified using 10%Pd/C (wet). Similar results are also obtained with 5%Pd/C, also provided by way of example, further adding to the cost effectiveness of the process.
The second stage incorporates direct conversion of compound of formula (1) to Venlafaxine hydrochloride of formula (3) without chromatographic isolation of its free base (Venlafaxine) as suggested in prior art reference (EP 0112669) rendering the process industrially suited and commercially viable.
The yields of the final compound are high as compared to the prior art references.

The process of the present invention will be explained in more detail with reference to the following examples, which are provided by way of illustration only and should not be construed as limit to the scope of the reaction in any manner.

### EXAMPLE 1

### Preparation of 1-[cyano(p-methoxy phenyl)methyl]cyclohexanol

Methanol (400 ml) was cooled to 0-5° C. and sodium methoxide (88 g) was added to the methanol slowly while maintaining temperature between 0 and 15° C. After cooling the sodium methoxide solution to −2 to 5° C., 4-methoxy benzyl cyanide (80 g) was added slowly over 45-60 minutes. The reaction mixture was maintained at 0-5° C. for 2 hrs and cooled to −5-2° C. Then cyclohexanone (70 g) was added to the reaction mixture over 60-90 minutes, and the resulting reaction mixture was maintained at 0-5° C. for 4-5 hours. Water (800 ml) was added while maintaining the temperature between 0 and 8° C. After 30-45 minutes, the crude material was filtered and washed with water (80 ml). The wet cake was added to toluene (800 ml), and the mixture was heated to about 40-50° C. to get a clear solution. The organic layer was separated at the same temperature and subsequently dried with anhydrous sodium sulphate (10 g). After the toluene solution is filtrated, the filtrate was cooled to 5-10°C. and maintained at the same temperature for 2 hours. Precipitated solids were filtered, washed with toluene (30 ml), and dried at 50-60° C. under vacuum to give the desired product (114-118 g). The product can be further purified by recrystallization in toluene.

### EXAMPLE 2

### Recrystallization of 1-[cyano(p-methoxy phenyl)methyl]cyclohexanol

To toluene (185 ml) was added 1-[cyano(p-methoxy phenyl)methyl]cyclohexanol (70 mg). The mixture was heated to 80-90° C., and any insoluble particles were removed by filtration at the same temperature. The filtrate was cooled to 0-10° C. and maintained at the same temperature for 1-2 hours. Crystallized solids were filtered, washed with toluene (18 ml), and dried at 50-60° C. to give the titled product (59.6-66.5 g; 85 -95%)

### EXAMPLE 3

### Preparation of acetic acid salt of 1-[2-amino-1-(4-methoxyphenyl)ethyl;]cyclohexanol

Acetic acid (360 ml) and 1-[cyano(p-methoxy phenyl)methyl]cyclohexanol (60 g) were added into dried auto clave vessel, into which 10% Pd/C (50% wet, 3.6 g) was added and H₂ gas was flushed out three times with pressure of 2 kgs/cm². While supplying H₂ gas at 0-17 kg/cm², the mixture was slowly heated to 50° C. and then heated to 50-55° C. for about 10-12 hours with H₂ pressure of 15-17 kg/cm². After confirming the completion of the reaction with thin liquid chromatography, the mixture was cooled to 25-35° C., and the pressure was released slowly. The catalyst was filtered off with help of acetic acid (60 ml), and then the acetic acid of the filtrate was distilled of completely under vacuum below 70° C. To the residue, water (60 ml) and methylene chloride (300 ml) were added at 25-30° C., and the mixture was cooled to 0-10° C. Ammonia solution (240 ml) was added and the mixture was stirred for 10-20 minutes at the same temperature. The organic layer was separated. The aqueous layer was extracted again with methylene chloride (120 ml), and the organic layers were combined. After the methylene chloride was distilled off completely, isopropanol (30 ml) was added and distilled off completely again. The residue was cooled to 25-35° C., and isopropanol (120 ml) and n-heptane (240 ml) were added. The resulting mixture was maintained at 0-5° C. for 1-2 hours, and solids were filtered and washed with n-heptane (60 ml). The isolated wet compound was added to ethyl acetate (30 ml), which was distilled off completely under vacuum. To the residue was added ethyl acetate (360 ml). The resulting solution was cooled to 0-10° C., and acetic acid (21 ml) was added slowly. After stirring the reaction mass at 0-10° C. for 1-2 hours, the compound was filtered, washed with ethyl acetate (30 ml), and dried at 50-60° C. to give the titled compound (34-41.5 g; 45-55%).

### EXAMPLE 4

### Preparation of 1-[2-Amino-1-(4-methoxy phenyl)ethyl]cyclohexanol (Formula-1)

A suspension of 1-[cyano-(4-methoxy phenyl)methyl]cyclohexanol (60 g) of formula (2) in acetic acid (360.0 ml) was hydrogenated in an autoclave at a pressure of 10-15 kg/cm2 in presence of 10% (50% wet) palladium on charcoal (2.4 g) at a temperature of in the range of 50-55° C. The same temperature and pressure was maintained till the hydrogenation was substantially complete, after which the catalyst was filtered accompanied by washings with acetic acid (60.0 ml). The filtrate and washings were combined and evaporated under reduced pressure. The resultant solid residues were dissolved in 60 ml of water and basify with 240 ml of ammonia and extracted with methylene chloride (300 ml and 120 ml respectively). Evaporate the combined extracts of the organic solvent completely under reduced pressure to get oily residue. Dissolve the oily residue in 180ml of isopropanol and cool to 0° C. Filter the separated desired compound, this low melting solid was taken into 300 ml ethyl aecetate and added 28 ml of acetic acid at 0° C. Filter the separated compound of formula (1) 31.5 gr

### EXAMPLE 5

### Preparation of 1-[2-(dimethylamino)-1-(4-methoxy phenyl)ethyl]cyclohexanol hydrochloride (Venlafaxine hydrochloride of Formula-3)

A stirred mixture of 1-[2-amino-1-(4-methoxy phenyl) ethyl]cyclohexanol (55.0 g), formic acid (25.0 ml), 40% formaldehyde solution (92.0 ml) and water (275.0 ml) was heated at 90-98° C. for 19 hrs. The reaction mass was cooled and washed with chloroform (4×55.0 ml). The washings were discarded. The aqueous layer was then cooled to 5° C. and basified with 48% sodium hydroxide solution (25.0 ml). The product was extracted from the alkaline aqueous layer by chloroform (3×100.0 ml). The organic layer was then evaporated under reduced pressure to yield an oily residue, which was dissolved in isopropyl alcohol (225.0 ml). The resultant solution was acidified with isopropyl alcohol hydrochloride till pH of ˜2 were achieved. The precipitated solid was filtered and washed with isopropyl alcohol (25.0 ml). It was then dried at 55-60° C. to yield the desired compound of formula I (Yield: 44.0 g).

1. An improved process for the preparation of compound of formula (1), which comprises:
a. reduction of 1-[cyano(4-methoxyphenyl)methyl)cyclohexanol of formula (2) with
palladium on charcoal in an organic acid selected from formic acid, acetic acid or
propionic acid, preferably acetic acid in an autoclave at a pressure of 5-25 kg/cm2
preferably 10-15 kg/cm2 at a temperature in the range of 30-75° C., preferably at 50-55° C. till the hydrogenation substantially complete;
b. filteration of the palladium catalyst from the reaction mass of step (a);
c. evaporating the filtrate of step (b) under reduced pressure;
d. suspending the solid type residue of step (c) in water and the resulted solution was basified by addition of aqueous ammonia;
e. extraction of the solution of step (d) with halogenated hydrocarbon solvents such as methylene chloride or chloroform preferably methylene chloride;
f. washing the organic solution of step (e) with water;
g. evaporation of the organic solvent from step (f) under reduced pressure to get residue;
h. dissolution of the residue from step (g) in C1-C4 alcohols preferably isopropanol;
i. cooling the mass of step (h) at 0-35° C. preferably at 0-5° C.;
j. filtration of separated solid from step (i) to get the desired compound of formula (1).
k. taken the low melting solid obtained from step (j) into ethyl acetate and added acetic acid at 0° C.;
l. filtered the separated solid from step (k), and dried at 45-60° C. preferably 55° C.

2. A process according to claim 1 step (a), palladium on charcoal can be used as 5% or 10%.

3. A process for the preparaion of Venlafaxine hydrochloride without isolation of its freebase by conversion of compound of formula (1) prepared as per the claim-1.

4. An improved process for the preparation of 1-[2-amino-1-(4-methoxy phenyl)ethyl]cyclohexanol (formula-1) and further its conversion to Venlafaxine hydrochloride without isolation of freebase is substantially as herein described and exemplified.
